(19) United States
(12) Patent Application Publication
(10) Pub. No.: US 2004/0143221 A1
(43) Pub. Date: Jul. 22, 2004

(54) Biomedical implant for sustained agent release

(76) Inventor: John H. Shadduck, Tiburon, CA (US)

Correspondence Address: John H. Shadduck, 1490 Vistazo West, Tiburon, CA, 94920, US

(21) Appl. No.: 10/748,909

(22) Filed: Dec. 27, 2003

Related U.S. Application Data
(60) Provisional application No. 60/436,840, filed on Dec. 27, 2002.

(51) Int. Cl. A61M 5/32

(52) U.S. Cl. 604/175; 424/422

(57) ABSTRACT

A biomedical implant for sustained local delivery of at least one pharmacologically active agent to the duodenum and small intestine of the GI tract. In one embodiment, the implant body comprises and elongate flexible, biodegradable polymeric element that is attached to the wall of the duodenum after introduction through a gastroscope. Biodegradation of the polymeric releases the pharmacologically active agent. Another embodiment of the implant carries transducer means for monitoring at least one physiological parameter of the targeted site, such as the acidity-alkalinity level. The drug-eluting implant body can carry and release agents such antibiotics and antibacterial agents, anti-fungal agents, anti-viral agents, anti-allergenics, anesthetics, analgesics, anti-cancer agents, immunological response modifiers and any other drug for treating irritable bowel syndrome (IBS) or other disorders requiring sustained agent release.

### BACKGROUND OF THE INVENTION

1. Field of the Invention

The present invention relates to an agent-release implant for implantation in GI tract for sustained agent delivery to the region of the duodenum and small intestine. More in particular, one type of implant includes drug-release capabilities and an optional monitoring system for treating irritable bowel syndrome (IBS). The implant body locally delivers a pharmacologically active agent to the small intestine, and can modulate the rate of agent delivery in response to a physiological parameter of the tract, such as an acidity-alkalinity level that is believed to reliable indicator of IBS symptom onset.

2. Description of the Related Art

Irritable bowel syndrome (IBS) is a chronic medical disorder characterized by (i) abdominal pain which is a sine qua non for diagnosis, and (ii) altered bowel function. These two symptom components will characterize a diagnosis of IBS. A number of epidemiological studies suggest very high incidence of IBS across the globe—with as much as about 10% to 20% of the population of the United States and Canada being affected. IBS is a disease of the young and middle-aged, with incidence falling at beyond about 60 years of age. The disorder is most common among females. The symptom complex of IBS is characterized by abdominal discomfort associated with altered bowel habits. In addition, there is a substantial psychological co-morbidity in many patients. At the same time, there are frequent extra-intestinal manifestations associated with poor sleep, fatigue, changes in libido, loss of energy, and a number of other symptoms that often overlap with symptoms in the gut, as well as outside of the colon, such as dyspeptic symptoms or non-cardiac chest pain. It is also known that many IBS patients have other symptoms including irritable bladder, chronic fatigue, fibromyalgia and headaches. This is related to a number of abnormalities, some of them having to do with autonomic dysregulation and visceral hypersensitivity, altered pain modulation, abnormal health-seeking behavior and, to some degree, iatrogenesis. In post-infectious IBS patients, there is accelerated gut transit in many patients, increased visceral sensitivity, and evidence for alteration of intestinal permeability which relates to chronic diarrhea in many patients.

If one analyzed the prevalence of IBS when compared with other chronic medical diseases, IBS is a significant problem. IBS has an equal or slightly higher incidence than hypertension which is a very large well recognized medical problem. IBS is more common than asthma, diabetes and heart disease. IBS is more common than gastroesophageal reflux disease (GERD) which is a very common disorder. IBS represents about 12% of all patients seen in primary care practice, and as much as about 28% of patients in GI practices. In any form of office-based medical practice, IBS is an extremely common problem without adequate treatment options.

In a study by Gralnek published in Gastroenterology in 2000, the health-related quality of life was examined in patients with a number of medical diseases, both GI and non-GI related. When compared with GERD and diabetes in the baseline population, IBS patients had by far the lowest quality of life. Thus, IBS is a condition that has a very low mortality—but a very high morbidity with much of the morbidity being nonmedical. The disease has very high socio-economic burdens besides the direct medical costs.

In another study, the number of IBS patients that missed work was out 30%; the IBS patients that cut back on their productivity at work was close to 50%. As many 12% of IBS patients changed jobs to allow work at home. Many IBS sufferers completely removed themselves from the extramural workforce. As many as 12% or the afflicted population lost jobs due to IBS.

The exact direct costs of the disease are difficult to determine. The indirect costs are reported to be around $20 billion, with the direct costs for medical care between $1.7 and $10 billion depending upon which study is relied upon for data. Many variables can be used in determining the cost of delivering IBS-related healthcare service in the U.S. For example, patients with IBS have approximately twice as many surgeries as non-IBS patients. Hysterectomies, appendectomies, and cholecystectomies are all more common in patients with IBS. Some estimates of the total direct and indirect costs for IBS are in the $30 billion range per year in the United States—which is a tremendous societal cost for what has been perceived as a disorder of lesser importance.

In summary, the impact of IBS is a multispectral, multidimensional problem in the U.S. Improved systems and methods are needed for reducing the high annual costs of the disease, and for limiting future increases in IBS incidence. The health care system needs to find ways to alleviate the symptoms of IBS patients and to reduce their level of healthcare utilization.

### SUMMARY OF THE INVENTION

In general, the biomedical implant corresponding to the invention comprises a controlled-release implant body that permits either extended constant rate delivery, extended and intermittent delivery, or both delayed and extended delivery of one or more pharmacologically active agents into the upper part of the small intestine. Such an implant, it is believed, will be important for treating irritable bowel syndrome in that various small molecule drugs are being investigated for treating IBS symptoms. More than 70% of small molecule drugs are absorbed almost exclusively in the small intestine wherein the transit time duration is limited to 2 to 4 hours. The implant is particularly adapted for greatly extending the time of action of these classes of drugs, wherein it is necessary to increase the duration of absorption by maintaining agent delivery in the duodenum and/or small intestine. A significant proportion of nutrients, drugs, vitamin and the like are absorbed in the small intestine, and more particularly in the duodenum which consists of the first several inches of the gastrointestinal tract beyond the stomach. In the duodenum, in addition to water, mucus and electrolytes, secretions from the liver and pancreas join secretions from the intestinal mucosa to facilitate digestion and nutrient and agent absorption. The anatomy of the small intestines is unique in that very large surface area membranes are presented to provide better absorption. The lining of the small intestines is composed of many villi, or finger like projections, which extend even more as projections called the brush border. The area is highly perfused with blood. These factors contribute to the very high surface area, increasing the likelihood of drug absorption taking place, if the ionization criterion is met. The pH can reach 7 to 8 in this area. Still, the intestinal epithelium may form a permeability barrier for absorption of orally administered drugs. Drugs mainly pass this intestinal epithelium barrier by two routes: the transcellular pathway (across the cell membranes) and the paracellular pathway (i.e. through the intercellular spaces between the intestinal cells). For many drugs, the predominant route of transport across the intestinal epithelium is the transcellular route, whereas small hydrophilic compounds and peptides are mainly transported via the paracellular route. Additionally, the intestinal absorption of drugs can be reduced by the presence of efflux proteins and/or metabolism in the gut. The implant of the invention provides a sustained release of agent to maximize the absorption of agents by both the transcellular and paracellular pathways.

The implant and method of the invention comprise an elongated flexible polymer body that can be attached to the wall of the GI tract in the region of the duodenum. The implant body carries one or more drugs within the polymer matrix that are released in a controlled manner by the biodegradation of the exposed surfaces of the implant. The implant is introduced to the targeted site in a working channel of a gastroscope and includes attachment means for tethering an end of the implant to the wall or the GI lumen. In one embodiment, the polymer component of the implant body is at least partly a shape memory polymer that has a temporary compacted shape for introduction and an elongated memory shape. The drug-eluting implant body can carry and release agents such antibiotics and antibacterial agents, anti-fungal agents, antiviral agents, anti-allergenics, anesthetics, analgesics, anti-cancer agents, immunological response modifiers and any other drug for treating IBS or any other disorder.

### BRIEF DESCRIPTION OF THE DRAWINGS

FIG. 1 is a cut-away view of a patient's lower stomach, duodenal bulb, duodenum and jejunum illustrating a Type “A” implant corresponding to the invention.

FIG. 2A is a perspective view of the Type “A” implant of FIG. 1 in a pre-deployed state being introduced from a working channel in an endoscope.

FIG. 2B is an enlarged view of an exemplary attachment portion of the implant body of FIG. 1.

FIG. 3 is a cut-away view of a portion of a patient's GI tract with an alternative elongated Type “A” implant of the invention deployed in the GI tract.

FIG. 4 is a cut-away view of a patient's lower stomach and duodenum illustrating a Type “B” implant that carries a transducer system corresponding to an alternative embodiment of the invention.

### DETAILED DESCRIPTION OF THE INVENTION

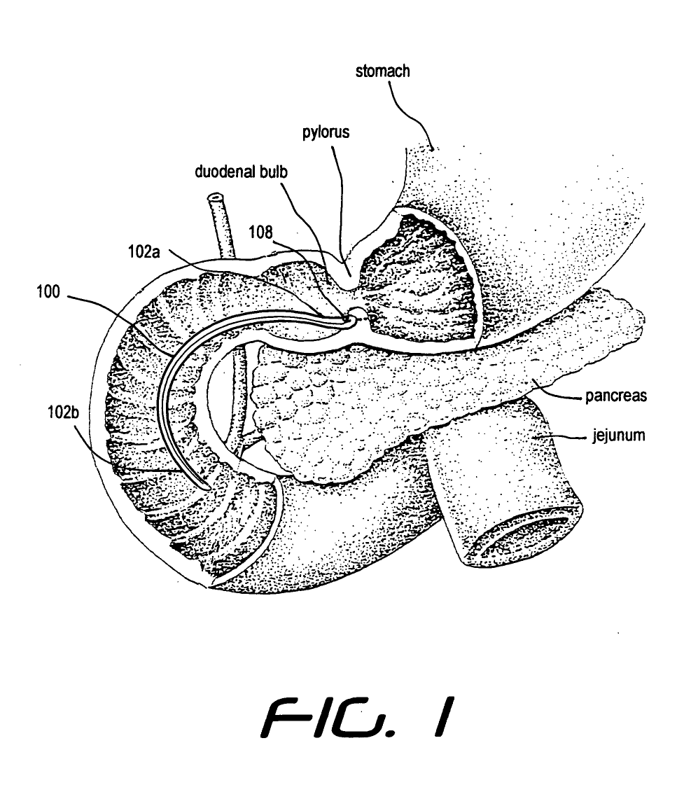

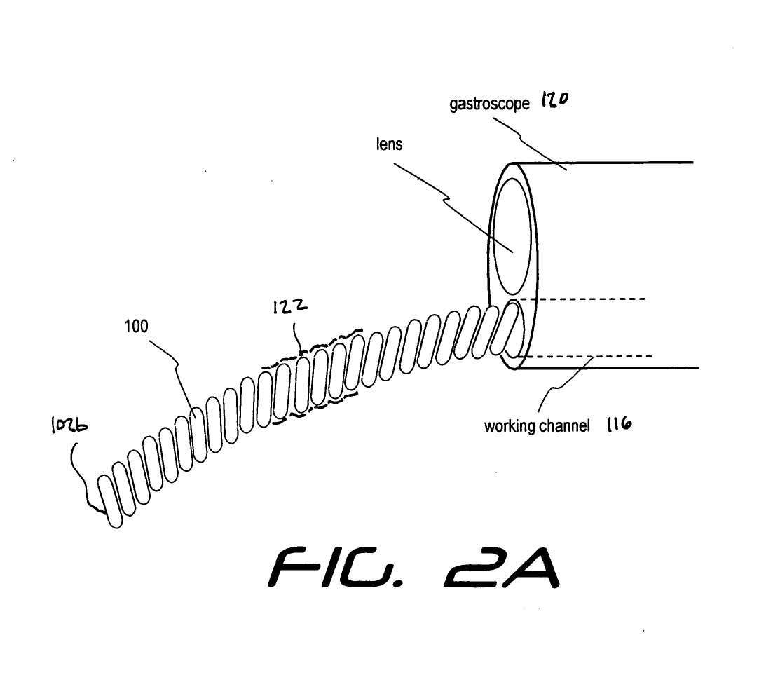

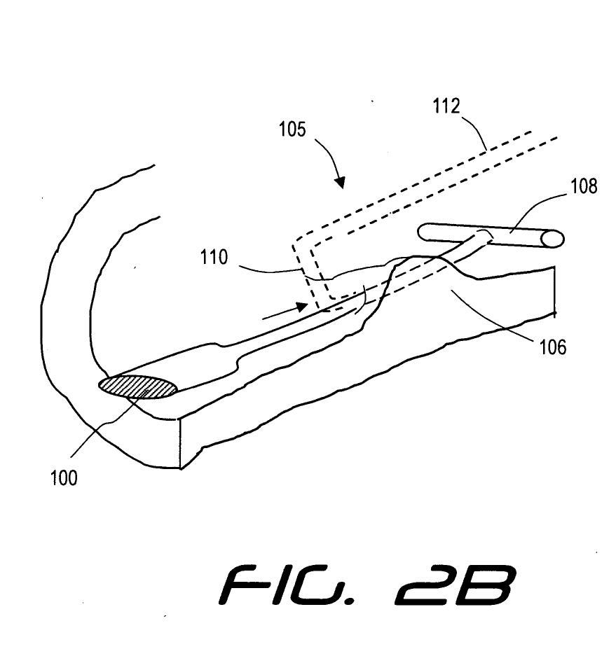

1. Type “A” implant for treating IBS. FIGS. 1 and 2 illustrate a Type “A” implant 100 corresponding to the invention wherein the implant body 100 is deployed in a patient's GI tract. The implant body defines first and second ends 102a and 102b wherein the first end carries attachment means indicated generally at 105 for attaching the implant to the wall of the lower stomach, pylorus, duodenal bulb, duodenum or other region of the intestinal tract. The attachment means 105 can be any suitable mechanism known in the art (not limiting) and may be a deformable hammer-anvil staple-type member, a spring-clip fastener member, a polymer clip, a helically-formed fastener, an adhesive fastener, a band-type mechanism for disposition around a tissue protrusion (e.g., an elastic band mechanism), a barb-tipped member, a toggle-tipped penetrating member or a thermal-energy based tissue adhering member.

The implant 100 of FIG. 1 is introduced through the working channel of a gastroscope (see FIG. 2A) together with a tool means for enabling the attachment of the implant with the attachment means 105. As can be seen in FIG. 1, in one embodiment and method of the invention, the implant body 100 is attached to the lumen wall 106 in the region of the pylorus or duodenum by a bioabsorbable polymer fastener portion 108. FIG. 2B illustrates the working end 110 of an introducer 112 that is adapted to insert the barbed or toggle end of a polymer fastener 108 through the targeted body wall 106 of the GI tract.

As can be seen in FIG. 2A, one embodiment of implant body 100 as in FIGS. 1 and 2A can be compacted into a smaller diameter state and/or coiled state for deployment through the working channel 116 of a gastroscope 120. In such an embodiment, the implant 100 is elongate and coiled and can be retained in the coiled state by a rapidly biodegradable polymer sheath portion 122 that encases the implant 100 to provide its temporary shape. In a related embodiment, the implant 100 of FIG. 1 is of a bioabsorbable shape memory polymer (SMP) that has a first temporary shape for introduction that expands to an increased cross-section and/or length after implantation. A background on shape memory polymers is provided below.

One objective of the invention is the controlled delivery of a pharmacological agent over an extended period to the targeted GI site consisting of the duodenum and small intestine. The delivery of agents for absorption in the upper GI tract over an extended period is a significant challenge, since the transit time through the small intestine for orally administered drugs can be very rapid. In the case of IBS treatments, the implant 100 of FIG. 1 can provide local, sustained or intermittent agent release over an extended time period. Oral drug delivery for treating local IBS conditions is not optimal since often the drug will be limited in solubility and be poorly absorbed. Any drugs that may be more freely absorbed may be processed by the patient's system rapidly that the agent's time of action will be short requiring the patient to take frequent doses. In light of the wide range of different physical, chemical, pharmacological and pharmacokinetic characteristics of drugs that may be utilized for treating IBS, it is believed that a local agent-eluting implant system will be optimal for IBS treatments for practically any of the chemotherapeutic agents being evaluated for IBS.

One preferred agent delivery system is the use of an implant body of a biodegradable polymer that carries a pharmacologically active agent therein. In such a biodegradable polymer implant, the bioactive agent is released as the polymer degrades or dissolves, making the agent readily available to the GI tract environment. Bioerodable drug-eluting polymers are known in the field of endovascular stents and ocular implants. Bioerodible or biodegradable polymers suitable for implant body 100 can be designed to modulate, regulate, enhance or sustain the release rate of the pharmacologically active agent, and for example can be polymer types and systems as described in U.S. Pat. Nos: 6,596,296; 5,527,801; 6,358,556; 6,284,305; 6,099,562; 5,873,904; 5,342,348; 5,707,385; 5,824,048 and 6,013,853 which are incorporated herein by reference. Other polymers and processes for preparing the polymers are well known, for example as disclosed in U.S. Pat. Nos: 4,304,765; 4,668,506; 4,959,217; 4,144,317; 5,824,074 and in Encyclopedia of Polymer Science and Technology, Vol. 3, published by Interscience Publishers, Inc., New York, and in Handbook of Common Polymers by Scott, J. R. and Roff, W. J., published by CRC Press, Cleveland, Ohio. Many of the biodegradable and absorbable polymers known in the art use a derivation of five different building blocks, namely, glycolic acid, lactic acid, trimethylene carbonate, p-dioxanone, and caprolactone, any of which may be suitable for the implant body of the invention. The biodegradable polymer can be adapted for a constant release rate of a single agent over an extended time period, or more than one agent over differing time periods at different release rates based on different biodegradable layers or different biodegradable portions over the elongated length of the implant body.

Another type of bioabsorbable, custom tailorable polymer that can be used for implant 100 is a composition called a polyhydroxyalkanoate or a PHA polymer. These polymers are synthesized in nature by several microorganisms, and have been recognized as another class of naturally occurring biopolymers, along with the polyamino acids, polynucleic acids, polysaccharides, and polyisoprenoids. Unlike the other naturally occurring biological polymers, however, the PHA polymers are defined as a thermoplastic—a composition that repeatedly softens when heated and hardens when cooled. As such, these polymers can be processed much like conventional synthetic plastics. Until recently, the widespread use of PHA materials had been limited by the available production technology. In the 1980's, researchers at the Massachusetts Institute of Technology made a breakthrough in the development of new PHA production systems, when they successfully isolated the genes responsible for making these polymers in microorganisms. Metabolix, Inc. thereafter developed transgenic systems for the commercial production of PHA polymers, and successfully developed several transgenic fermentation methods for producing different PHA polymers. Currently, a library of different hydroxy acid building blocks can be accessed to tailor the desired properties of a PHA polymer. Using this approach, it is possible to make PHA polymers with mechanical properties spanning wide ranges, from high modulus to elastomeric, flexible to stiff, and weak to strong. Moreover, it is also possible to produce PHA polymers incorporating additional functionality thus allowing for easy derivatization of the materials. In addition to being able to tailor the mechanical and thermal properties of PHA polymers, it is also possible to use the library of building blocks to engineer desirable rates of bioabsorption in the body which can range from weeks to years. The following patents and publications are incorporated herein by reference in their entirety: Polyhydroxyalkanoate compositions having controlled degradation rates, Martin, D. P. et al., WO 99/32536 (Published July, 1999); Biological systems for manufacture of polyhydroxyalkanoate polymers containing 4-hydroxyacids, Huisman, G. W. et al, WO 99/14313 (Published March, 1999); Polyhydroxyalkanoates for in vivo applications, Williams, S. F. et al., WO 98/51812 (Published November, 1998); Metabolic engineering of poly(3-hydroxyalkanoates): From DNA to plastic, Madison, L. L. and Huisman, G. W. Microbiol. Mol. Biol. Rev. 63:21-53 (1999); and Tissue engineering of heart valves—Early in vitro experiences with a polyhydroxyalkanoate biopolyester as a scaffold, Sodian, R. et al., Abstract from the Meeting of the American Society for Artificial Internal Organs, June 2-5, 1999, San Diego, Calif. The polyhydroxyalkanoates used in the invention are being developed by, and can be acquired from, Tepha, Inc., 303 Third Street, Cambridge, Mass. 02142.

The polymer agent release means of the system can be adapted to carry any agents known in the art for treating symptoms of IBS, including (not limiting): agents with anti-inflammatory properties, agents for modulating serotoninergic pathways, agents for modulating acidity-alkalinity levels, anti-fungal agents, antibiotics, bactericides and neuro-modulation agents. In IBS patients, there is clear evidence that many patients have increased presence of enterochromaffin (EC) cells in the colon. This evidence suggests the possibility of increased release of serotonin (5-HT) in such patients. Serotonin is involved almost every level of the communication between the gut and brain, both going from gut to the brain and then from brain to gut. Thus, one appealing therapeutic avenue is to focus on the site where most of the 5-HT is being released and try to affect outcome and symptoms by modulating symptoms at the level of the gut trying to avoid the possible side effects that may come with more central modulation of serotoninergic pathways.

As described briefly above, a shape memory polymer (SMP) can be uses in a base material of the implant body 100. As background, the class of shape memory polymers (SMPs) comprises a type of co-polymer that consists of a hard segment and a soft segment each having a different glass transition temperature. One segment can have a glass transition temperature ranging between about 35° C. and 80° C. at which the shape memory polymer changes from a first dimension or volume to a second dimension or volume. For example upon deployment in tissue, one segment of the polymer can have a glass transition temperature of about 35° C. to 37° so that body temperature causes the implant to move from an initial compacted position to an expanded position. Such shape memory polymers (SMPs) demonstrate the phenomena of shape memory based on fabricating a segregated linear block co-polymer, typically of a hard segment and a soft segment. The shape memory polymer generally is characterized as defining phases that result from glass transition temperatures in the hard and a soft segment. The hard segment of SMP typically is crystalline with a defined melting point, and the soft segment is typically amorphous, with another defined transition temperature. In some embodiments, these characteristics may be reversed together with the segment's glass transition temperatures.

In one implant embodiment, when the SMP material is elevated in temperature above the melting point or glass transition temperature of the hard segment, the material then can be formed into a memory shape. The selected shape is memorized by cooling the SMP below the melting point or glass transition temperature of the hard segment. When the shaped SMP is cooled below the melting point or glass transition temperature of the soft segment while the shape is deformed, that temporary shape will be fixed. The original shape is recovered by heating the material above the melting point or glass transition temperature of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. in many embodiments—or a higher selected temperature when the implant body is adapted to cooperate with magnetic responsive particles in the polymer that cooperate with a remote energy source.

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. The scope of the invention of using SMPs in implants extends to the control of such physical properties within the implant for numerous therapeutic applications.

Examples of polymers that have been utilized in hard and soft segments of SMPs include polyethers, polyacrylates, polyamides, polysiloxanes, polyurethanes, polyether amides, polyether esters, and urethanebutadiene copolymers. See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al, all of which are incorporated herein by reference. SMPs are also described in the literature: Ohand Gorden, Applications of Shape Memory Polyurethanes, Proceedings of the First International Conference on Shape Memory and Superelastic Technologies, SMST International Committee, pp. 115-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque C1) 6:377-84 (1996))(all of the cited literature incorporated herein by this reference).

In another embodiment, the implant body can be a foamed SMP or CHEM (cold hibernated elastic memory material) that possesses several potential advantages, for example: very large shape recovery strains are achievable (i.e., a substantially large reversible reduction of the Young's Modulus in the material's rubbery state); the material's ability to undergo reversible inelastic strains of greater than 10%, and preferably greater that 20% (and up to about 200%-400%); shape recovery can be designed at a selected temperature between about 30° C. and 45° C. which may be useful for the implants. These polymers also demonstrate unique properties in terms of capacity to alter the material's water or fluid permeability and thermal expansivity. However, the material's reversible inelastic strain capabilities leads to its most important property-the shape memory effect. If the polymer is strained into a new shape at a high temperature (above the glass transition temperature T_{S}) and then cooled it becomes fixed into the new temporary shape. The initial memory shape can be recovered by reheating the foam above its T_{S}. The shape memory foams are of particular interest for various implants because they provide even lower density than solid SMPs.

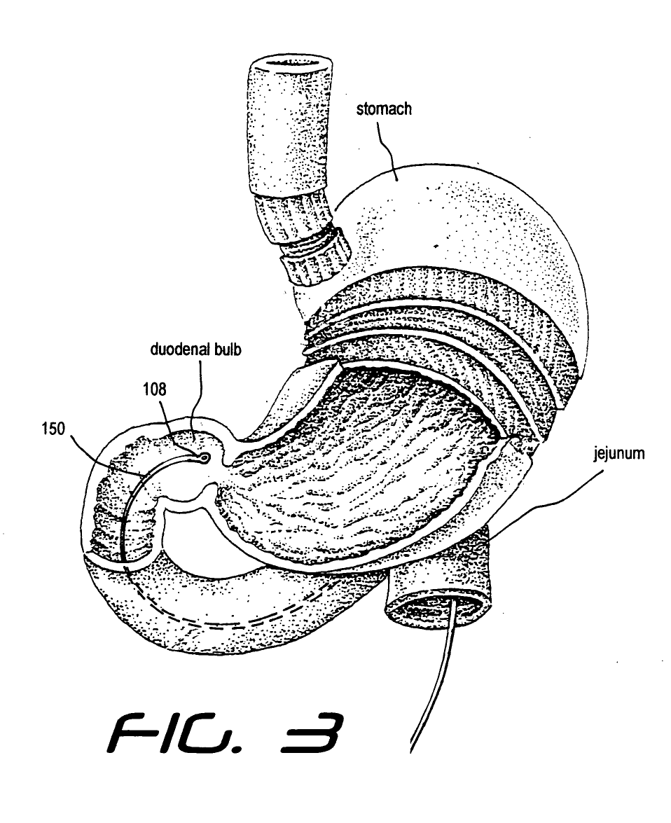

FIG. 3 shows an alternative implant 150 wherein the implant body is very elongate, and can extend from about 50 mm. to 500 mm. or longer. The implant 150 can be of a shape memory polymer that initially has a compacted, shortened temporary shape. Following implantation, the SMP implant 150 can be actuated by thermal means or acidity to move to its memory shape which is highly elongated. The cross-section of the implant member can decrease in the distal direction. Also the flexibility of the elongate implant can increase in the distal direction. These properties can assist in preventing elongate implants from becoming entangled. The implant would then be atraumatically disposed within the intestinal lumen and bear some resemblance to a tapeworm. The cross-section may be round, oval, rectangular or any other shape. The rate of biodegradability also may vary over the length of the implant body. The implant also can be fabricated of a hydrogel known in the art of a type known to vary its permeability, or bioabsorption rate in response to changes in Ph levels to allow intermittent agent delivery.

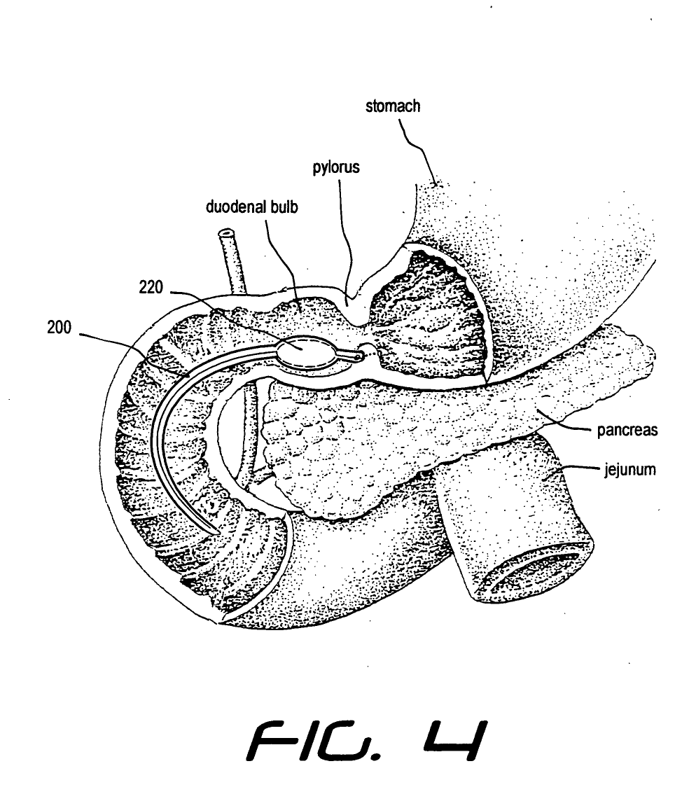

2. Type “B” implant for treating IBS. FIG. 4 illustrates a Type “B” implant 200 corresponding to the invention wherein the implant body 200 again is deployed in a patient's GI tract—preferably at a targeted site in the region of the duodenum. This polymer implant body 200 carries all of the agent-delivery functionality of the Type “A” embodiment and further carries transducer means indicated at 220 for obtaining data on a selected physiological parameter about the targeted site. The implant further can carry data transmission means for transmitting the data to an external monitoring system. A transducer system with remote monitoring for treating severe heartburn (GERD or gastroesophageal reflux disease) was proposed in U.S. Pat. No. 6,285,897 to Kilcoyne et al.

The implant 200 of the invention can carry an acidity-alkalinity transducer system or a transducer for measuring pressure and/or temperature. Further, micro-array technology is being developed that can be used to measuring bio-parameters which can be used in the invention. The system can have integrated feedback mechanisms for agent release based on the monitored parameters. For example, remote actuation technologies are known in the art for release of agents from a micro-reservoir by means of an of electrically sacrificial reservoir port closure. The transducer and data transmission means typically can be carried within a capsule that will pass through the patient's GI tract following controlled biodegradation of the implant or its attachment means.

It should be appreciated that the implant can be utilized for treating disorders and diseases besides IBS wherein sustained agent release directly to the duodenum and small intestine are preferred over oral or other drug delivery means. For example, a wide variety of drugs can be delivered by the implant system, for example, from the following classes: antibiotics (e.g. tetracycline, chlortetracycline, bacitracin, neomycin, polymyxin, gramicidin, oxytetracycline, chloramphenicol, gentamycin, and erythromycin); antibacterial agents (e.g., sulfonamides, sulfacetamide, sulfamethizole and sulfisoxazole); anti-fungal agents (e.g., such as fluconazole, nitrofurazone, amphotericine B, ketoconazole, and related compounds); anti-viral agents (e.g., trifluorothymidine, acyclovir, ganciclovir, AZT, foscamet, vidarabine, trifluorouridine, ribavirin); anti-allergenics (e.g., methapyriline; chlorpheniramine, pyrilamine and prophenpyridamine); anti-inflammatories (e.g., hydrocortisone, dexamethasone, fluocinolone, prednisone, prednisolone, methylprednisolone, fluorometholone, betamethasone and triamcinolone); anesthetics; analgesics; cell transport/mobility impending agents; decongestants (e.g., phenylephrine, naphazoline, and tetrahydrazoline); cell cycle inhibitors, gene therapy compounds; miotics; anticlotting agents; anti-diabetic agents; anti-cancer agents; immunosuppresants or immunological response modifiers; hormones; peptides; nucleic acids and other macromolecules or a combination thereof. Practically any drug may be delivered with the implant of the invention, and there are no particular restrictions in terms of molecular weight etc. It should be appreciated that the implant also can be any dimension for implantation in any body lumen.

Those skilled in the art will appreciate that the exemplary embodiments and descriptions thereof are merely illustrative of the invention as a whole. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention.

What is claimed is:

1. A method for drug delivery comprising the steps of providing an implantable agent-release body carrying at least one pharmacologically active agent, and implanting the agent-release body in a targeted site within the gastrointestinal (GI) tract for sustained release of the pharmacologically active agent in the region of the duodenum and small intestine.

2. A method as in claim 1 wherein the implanting step includes utilizing attachment means to attach the agent-release body to a wall of the GI tract.

3. A method as in claim 2 wherein the agent-release body is attached to a wall of the duodenum.

4. A method as in claim 1 wherein the implanting step includes deployment of the agent-release body from a first shape to a second shape.

5. A method as in claim 4 wherein the providing step includes fabricating the agent-release body at least partly of a biodegradable polymer.

6. A method as in claim 4 wherein the providing step includes fabricating the agent-release body at least partly of a shape memory polymer.

7. A method as in claim 1 wherein the agent-release body releases an agent selected from the class consisting of antibiotics, antibacterial agents, anti-fungal agents, anti-viral agents, anti-allergenics, anesthetics, analgesics, cell transport/mobility impending agents, decongestants, anti-diabetic agents, anti-cancer agents, immunosuppresants, immunological response modifiers, hormones, cell cycle inhibitors, gene therapy compounds, miotics, anticlotting agents, peptides and nucleic acids.

8. A biomedical implant comprising an elongated flexible implant body dimensioned for extending within the GI tract implantation from the region of duodenal bulb or duodenum, attachment means at an end portion of the implant body and at least one pharmacologically active composition within the implant body.

9. The biomedical implant wherein the implant body is at least partly of a biodegradable polymer.

10. The biomedical implant of claim 8 wherein the implant body comprises a biodegradable polymer with the pharmacologically active composition carried therein.

11. The biomedical implant wherein the implant body is at least partly of a shape memory polymer.

12. The biomedical implant of claim 9 wherein the biodegradable polymer is at least in part a hydrogel.

13. The biomedical implant of claim 8 wherein the attachment means is selected from the class consisting of a barb-tipped member, a toggle-tipped member, a staple, a spring clip, a polymer clip, a helically-formed fastener, an adhesive fastener, a thermal-energy adhering member and a band-type mechanism for disposition around a tissue protrusion.

14. The biomedical implant of claim 11 wherein the implant body defines a first temporary shape for introduction and a second memory shape for extending within the GI tract.

15. The biomedical implant of claim 8 wherein the pharmacologically active composition is selected from the class consisting of antibiotics, antibacterial agents, anti-fungal agents, anti-viral agents, anti-allergenics, anesthetics, analgesics, cell transport/mobility impending agents, decongestants, anti-diabetic agents, anti-cancer agents, immunosuppresants, immunological response modifiers, hormones, cell cycle inhibitors, gene therapy compounds, miotics, anticlotting agents, peptides and nucleic acids.

16. The biomedical implant of claim 8 further comprising transducer means in the implant body for acquisition of data concerning a physiological parameter about the duodenum or small intestine and transmission means for transmitting the data to an external monitoring system.

17. The biomedical implant of claim 16 wherein the transducer means and transmission means include a transponder system.

18. A drug-eluting implant comprising a first portion of a shape memory polymer (SMP) portion and a second portion of a pharmacologically active composition, the shape memory polymer capable of a temporary shape and a memory shape.

19. The drug-eluting implant as in claim 18 wherein the shape memory polymer has a compact temporary shape and an elongated memory shape.

20. The drug-eluting implant as in claim 18 further comprising a fastener portion for fastening an end of the implant the wall of a body lumen.
